(19) United States
(12) Patent Application Publication
(10) Pub. No.: US 2007/0015174 A1
(43) Pub. Date: Jan. 18, 2007

(54) High throughput formulation screening of drug candidates

(76) Inventors: Matthew Duan, Lisle, IL (US); Charles Douglas McDermott, Encinitas, CA (US); Aaron Ezra Hoffman, New Haven, CT (US); Lansing J. Stewart, Bainbridge Island, WA (US); Thorsteinn Thorsteinsson, Crest Hill, IL (US); Robert P. O'Fee, New Hope, PA (US); David E. Zembower, LaGrange, IL (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 11/339,294

(22) Filed: Jan. 25, 2006

Related U.S. Application Data
(60) Provisional application No. 60/647,951, filed on Jan. 28, 2005.

(51) Int. Cl. C12Q 1/68; G01N 33/53

(52) U.S. Cl. 435/6; 435/7.1

(57) ABSTRACT

Methods for screening compounds for their solubility and their solution stability in a variety of diverse formulations and sets of diverse formulations useful for screening compounds for their solubility and their solution stability.

### CROSS-REFERENCE TO RELATED APPLICATION

This application claims the benefit of U.S. patent application Ser. No. 60/647,951, filed Jan. 28, 2005, expressly incorporated herein by reference in its entirety.

### BACKGROUND

High throughput screening technology (HTS), genomics, and parallel synthesis in recent drug discovery programs yields increasing numbers of compounds for in vivo experiments in laboratory animals. Often these compounds have poor solubility and high lipophilicity because the use of organic solvents in HTS assays often disguises the fact that many of the hits are due to sparingly water solubility. Typically compounds with an aqueous solubility greater than 100 μ/mL are less likely to generate dissolution-related problems in development phase, but compounds with aqueous solubilities between 1 and 100 μ/mL may require a special formulation to overcome the poor absorption issues due to their low solubilities. Compounds having aqueous solubility less than 1 pg/mL present a real challenge to formulation. Glomme, A., et al., “Comparison of a Miniaturized Shake-Flask Solubility Method With Automated Potentiometric Acid/Base Titrations and Calculated Solubilities,” J Pharm. Sci. 94:1-16, 2005. Poor pharmacokinetics is the major reason why at least 40 out of 198 clinical candidates were terminated for development. This number could be even higher considering lack of efficacy (30% of development terminations) may be attributed to the undesirable pharmacokinetics, which could result in insufficient concentrations to afford a response at the site of action. Earlier structure-activity relationship (SAR) optimizations were focused exclusively on the receptor binding, which generates compounds that are poorly soluble, too lipophilic, and have higher molecular weights. Kennedy, T., “Managing The Drug Discovery/Development Interface,” Drug Discovery Today 2:436-444. 1997. Formulation of these poorly soluble compounds creates a major bottleneck for drug discovery and development. Formulation compounds into pharmaceutical accepted excipients is typically performed on an as-needed basis in order to progress compounds through initial pharmacokinetic assessment and further toxicity studies. Following these initial studies, formulation is routinely re-performed to develop marketable dosage form on development candidates.

In the early stages of discovery, efficacy metrics take precedence over most other types of assessment, particularly formulations. Yet how well a compound can be formulated has a major impact on the final product profile and its corresponding commercial value. Formulation at this early stage of discovery often encounters limited compounds, short timelines, and incomplete physicochemical characterization. It has been reported that there is typically have a 3-day window and 10 mg per discovery compound to formulate and deliver the vehicle information in order to stay within discovery timelines. Lee, Y. C., et al., “An Intravenous Formulation Decision Tree for Discovery Compound Formulation Development,” Int. J Pharm. 253:111-119, 2003. A rapid formulation screen has been developed to identify a potent cremophor-free formulation for paclitaxel. Such a formulation can be administrated in higher amounts given the elimination of the dose-limiting excipient. Chen, H., et al., “A High-Throughput Combinatorial Approach for the Discovery of a Cremophor El-Free Paclitaxel Formulation,” Pharm. Res. 20:1302-1308, 2003. The required drug amounts (3.5 g for paclitaxel and 184 mg for naproxen) during their formulation screenings is much greater than the typical available discovery compound (10 mg).

### SUMMARY

The present invention provides methods for screening compounds for their solubility and their solution stability in a variety of diverse formulations. A set of diverse excipients useful in the method is also provided.

In one aspect, the invention provides a method for screening compounds for solubility. In the method, a quantity of a compound is dispensed into each well of a plurality of wells to provide a plurality of wells each containing a quantity of the compound; an excipient is then added to each well; and the solubility of the compound in the excipient in each well is observed.

In one embodiment, the method includes the following steps:

(a) dispensing a quantity of a compound into each well of a plurality of wells to provide a plurality of wells each containing a quantity of the compound;

(b) capturing an image of each well containing the compound;

(c) adding a different excipient to each well;

(d) observing the solubility of the compound in the excipient in each well by capturing a second image of each well;

(e) heating the plurality of wells to a predetermined temperature for a predetermined time;

(f) observing the solubility of the compound in the excipient in each well by capturing a third image of each well;

(g) observing the solubility of the compound in the excipient in each well by capturing a fourth image of each well after a predetermined period of time; and

(h) comparing the first, second, third, and fourth images to evaluate the solubility of the compound in each well.

The method can further include observing the solubility of the compound in the excipient in each well by capturing a fifth image of each well after a predetermined period of time and comparing the first, second, third, fourth, and fifth images to evaluate the solubility of the compound in each well.

In another aspect of the invention, a set of formulation solutions is provided. The set of formulation solutions is representative of different solubilization approaches. Representative solubilization approaches include pH adjustment, co-solvent, oil, micelle, organic solvent/surfactant, complexation, microemulsion, self-emulsifying drug delivery system, and combinations thereof. Representative sets of formulation solutions comprising excipients are set forth in Tables 1 and 2.

By using sophisticated liquid handling and imaging hardware, informatics, and special plasticware, the present invention provides devised scaleable, high-throughput methods for simultaneously assessing compound solubility, solution stability, and crystal formation. The automated screening process utilizes only 5-10 mg of compounds to initially assess its performance in ninety-six (96) diverse formulations. In one embodiment, each experiment cycle takes three days to complete and that includes testing the solution stability, and crystallization of the compounds. The process is managed by relational database software that designs and creates optimization screens. The database software also controls the liquid handling, imaging, and plate hotel hardware. The invention provides a single system that can collect formulation data for more than two hundred (200) compounds per week, thus providing critical information to the pharmacologists, medicinal chemists, and pharmaceutical scientists on drug discovery and development teams.

### DESCRIPTION OF THE DRAWINGS

The foregoing aspects and many of the attendant advantages of this invention will become more readily appreciated as the same become better understood by reference to the following detailed description, when taken in conjunction with the accompanying drawing, wherein:

FIG. 1 is a bar graph illustrating the number of compounds from a sixty-four (64) compound set that were completely solubilized by each formulation in a representative formulation screening set consisting of the ninety-six (96) biocompatible formulations set forth in Table 1.

### DETAILED DESCRIPTION

The present invention provides methods for screening compounds for their solubility and their solution stability in a variety of diverse formulations. A set of diverse excipients useful in the method is also provided.

In one aspect, the invention provides a method for screening compounds for solubility. In the method, a quantity of a compound is dispensed into each well of a plurality of wells to provide a plurality of wells each containing a quantity of the compound; an excipient is added to each well; and the solubility of the compound in the excipient in each well is observed. In one embodiment, a different excipient is added to each well.

Observing the solubility of the compound in each well comprises capturing an image of each well. In one embodiment, an image is captured of the quantity of compound dispensed into each well before the addition of excipient.

In one embodiment, the plurality of wells is heated to a predetermined temperature and for a predetermined time after observing the solubility of the compound in each well. Following heating, the solubility of the compound is observed after heating the plurality of wells to a predetermined temperature and for a predetermined time. In one embodiment, the solubility of the compound is observed at one or more predetermined times after heating the plurality of wells.

In the method, the excipient is representative of a solubilization approach. Representative solubilization approaches include pH adjustment, co-solvent, oil, micelle, organic solvent/surfactant, complexation, microemulsion, self-emulsifying drug delivery system, and combinations thereof. In one embodiment, the excipient is selected from the excipients set forth in Table 1. In another embodiment, the excipient is selected from the excipients set forth in Table 2.

In one aspect, the present invention provides a method for screening compounds for solubility, comprising:

(a) dispensing a quantity of a compound into each well of a plurality of wells to provide a plurality of wells each containing a quantity of the compound;

(b) capturing an image of each well containing the compound;

(c) adding a different excipient to each well;

(d) observing the solubility of the compound in the excipient in each well by capturing a second image of each well;

(e) heating the plurality of wells to a predetermined temperature for a predetermined time;

(f) observing the solubility of the compound in the excipient in each well by capturing a third image of each well;

(g) observing the solubility of the compound in the excipient in each well by capturing a fourth image of each well after a predetermined period of time; and

(h) comparing the first, second, third, and fourth images to evaluate the solubility of the compound in each well.

The method can further include observing the solubility of the compound in the excipient in each well by capturing a fifth image of each well after a predetermined period of time and comparing the first, second, third, fourth, and fifth images to evaluate the solubility of the compound in each well.

In the method, a quantity of a compound is dispensed into each well of a plurality of wells to provide a plurality of wells each containing a quantity of the compound. The quantity of compound dispensed into each well can vary from about 5 to about 500 μg. In one embodiment, the quantity of compound dispensed is about 50 μg. In general, the quantity of compound dispensed into each well is the same. However, to evaluate a range of solubility targets for a compound, the quantity of compound dispensed can be varied (e.g., 5 μg, 10 μg, 25 μg, 50 μg).

As used herein, the term “compound” refers to a small molecule, a protein, a peptide, an oligonucleotide, and a nucleotide. In one embodiment, the compound is a therapeutic drug or therapeutic drug candidate. In one embodiment, the protein or peptide is an antibody or antibody fragment.

The plurality of wells can take a variety of forms. As used herein, the term “well” refers to any configuration that allows for the isolation of the compound and excipient for the duration of the solubility determination (e.g., heating and cooling) and allows for capturing of images of the compound and excipient combination over time.

In one embodiment, the plurality of wells is a plate housing the wells (e.g., a 96-well plate). The wells or plate housing the wells are made from a material that is impervious to the compounds and excipients used in the method. Each well has a volume sufficient to receive the compound and excipient, chemically stable with respect to the compound and excipient, and allows for capturing of an image of the compound and excipient combination over the duration of the solubility determination. A representative device useful in the invention is a polypropylene 96-well plate commercially available from Emerald BioSystems, Bainbridge Island, Wash., under the designation CLOVER crystallization plates. These plates are described in U.S. Pat. No. 6,039,804, expressly incorporated herein by reference in its entirety.

The compound can be dispensed to each well with a liquid handling system. A quantity of compound is first dissolved in a suitable solvent (e.g., a high vapor pressure organic solvent such a methanol) to provide a stock solution (e.g., 10 mg/mL). Then, a liquid handling system dispenses the appropriate volume (e.g., 5 μL) to each well to deliver the desired quantity of compound (e.g., 50 μg) to each well. A representative liquid handling system useful for dispensing the compound is commercially available from Emerald BioSystems, Bainbridge Island, Wash,, under the designation DROP MAKER. The liquid handling system is described in U.S. Pat. No. 6,818,060, expressly incorporated herein by reference in its entirety.

Once the solution of compound in suitable solvent is dispensed to the well, the solvent is evaporated to dryness to provide the compound in the well. Images of each well containing the compound are then captured. The images can be captured using a microscope/camera combination. A system useful for capturing and storing images taken during the method of the invention is commercially available from Emerald BioSystems, Bainbridge Island, Wash., under the designation CRYSTAL MONITOR. The system includes a stereomicroscope, a motorized stage, and a digital camera for capturing and storing high resolution images of individual wells. The system can further include a server-compatible relational database software that drives the individual hardware components and manages all data generated. Suitable software useful for the method of the invention is commercially available from Emerald BioSystems, Bainbridge Island, Wash., under the designation CRYSTAL MINER. The system and software is described in U.S. Pat. No. 6,811,608, expressly incorporated herein by reference in its entirety.

In the method, a volume of excipient is added to each well containing a quantity of compound. The volume of excipient added to each well can vary from about 1 to about 200 μL. In one embodiment, about 5 μL of excipient is added to each well. To evaluate a range of solubility targets for a compound, the volume of excipient added can be varied (e.g., 5 μL, 10 μL, 25 μL, 50 μL).

The excipient can be added to each well with a liquid handling system. A representative liquid handling system useful for dispensing the compound is commercially available from Emerald BioSystems, Bainbridge Island, Wash., under the designation MATRIX MAKER. The liquid handling system can be used to prepare the excipient stock solutions and for dispensing the appropriate volume of excipient to each well containing the compound. The system is suitable for accurately dispensing highly viscous excipients as well as high vapor pressure solvents. The liquid handling system is described in U.S. Pat. No. 6,818,060, expressly incorporated herein by reference in its entirety.

A representative formulation screening set composed of ninety-six (96) biocompatible excipients that are useful in the method of the invention is summarized in Table 1. The screening set includes excipients representative of the following solubilization approaches: pH adjustment, co-solvent, oil, micelle, organic solvent/surfactant, complexation, microemulsion, and self-emulsifying drug delivery system. Mixtures of excipients can also be used.

After adding each excipient to each well containing the compound, the solubility of the compound in the excipient in each well is observed by capturing a second image of each well. The image can be captured and stored by the system described above.

In one embodiment, the plurality of wells is heated to a predetermined temperature for a predetermined time after the addition of the excipient. The predetermined temperature can range from about 30 to about 65° C. In one embodiment, the predetermined temperature is 45° C. The predetermined time can range from about 5 to about 120 minutes. In one embodiment, the predetermined time is 30 minutes.

After cooling to ambient temperature, a second observance of the solubility of the compound in the excipient in each well is made by capturing a third image of each well.

After a predetermined time, the solubility of the compound in the excipient in each well is again observed by capturing a fourth image of each well. The predetermined time may range from one to more than one day. In one embodiment, the predetermined time is at least two days. In one embodiment, the predetermined time is three days.

The solubility of the compound in the various excipients is then evaluated by comparing the captured images (e.g., first, second, third, and fourth images). By comparing the images the compound's solubility in each excipient can be categorized as “not dissolved,” “partially dissolved,” “mostly dissolved,” “completely dissolved,” or “precipitate.” The collective information provides insight into the compound's formulation profile.

A representative method of the invention for screening triclocarban, an antibacterial agent, to obtain a solubility profile is described in Example 1.

In another aspect of the invention, sets of formulation solutions are provided. The sets of formulation solutions are representative of different solubilization approaches. Representative solubilization approaches include pH adjustment, co-solvent, oil, micelle, organic solvent/surfactant, complexation, microemulsion, self-emulsifying drug delivery system, and combinations thereof. Representative formulation screening sets composed of ninety-six (96) biocompatible excipients that are useful in the method of the invention are set forth in Tables 1 and 2. The compositions of the excipients and their applicability to oral and intravenous administration are also provided. The indication of single dose to obtain pharmacokinetics parameters for a compound (single PK) and intravenous dog friendliness (IV Dog friendly) is provided in Table 2.

TABLE 1
First Representative Formulation Screening Set.

| Condition | Excipient 1 | [%] | Excipient 2 | [%] | Excipient 3 | [%] | Excipient 4 | [%] | Oral | IV |
|---|---|---|---|---|---|---|---|---|---|---|
| 1 | NaH2PO4/phosphoric | | | | | | | | [symbol] | [symbol] |
| | acid, pH2 | | | | | | | | | |
| 2 | Na3 citrate/citric acid, pH3 | | | | | | | | [symbol] | [symbol] |
| 3 | Na acetate/acetic acid, pH5 | | | | | | | | [symbol] | [symbol] |
| 4 | NaH2PO4/Na2HPO4, pH7 | | | | | | | | [symbol] | [symbol] |
| 5 | NaH2PO4/Na2HPO4, pH8 | | | | | | | | [symbol] | [symbol] |
| 6 | Na2CO3/HCl, pH10 | | | | | | | | [symbol] | [symbol] |
| 7 | PEG 300 | 100% | | | | | | | [symbol] | |
| 8 | PEG 400 | 100% | | | | | | | [symbol] | |
| 9 | PG | 100% | | | | | | | [symbol] | |
| 10 | Glycerin | 100% | | | | | | | [symbol] | |
| 11 | Dimethylacetamide | 100% | | | | | | | [symbol] | |
| 12 | N-methyl-2-pyrrolidinone | 100% | | | | | | | [symbol] | |
| 13 | Polysorbate 80 | 100% | | | | | | | [symbol] | |
| 14 | Cremophor EL | 100% | | | | | | | [symbol] | |
| 15 | Softigen 767 | 100% | | | | | | | [symbol] | |
| 16 | Labrasol | 100% | | | | | | | [symbol] | |
| 17 | Labrafil M 1944 CS | 100% | | | | | | | [symbol] | |
| 18 | TPGS | 20% | | | | | | | [symbol] | |
| 19 | Povidone | 20% | | | | | | | [symbol] | |
| 20 | HPβCD | 40% | | | | | | | [symbol] | [symbol] |
| 21 | SBEβCD | 40% | | | | | | | [symbol] | [symbol] |
| 22 | RMβCD | 40% | | | | | | | [symbol] | |
| 23 | Corn oil | 100% | | | | | | | [symbol] | |
| 24 | Safflower oil | 100% | | | | | | | [symbol] | |
| 25 | Sesame oil | 100% | | | | | | | [symbol] | |
| 26 | Soybean oil | 100% | | | | | | | [symbol] | |
| 27 | Peppermint oil | 100% | | | | | | | [symbol] | |
| 28 | Oleic acid | 100% | | | | | | | [symbol] | |
| 29 | Neobee M-5 (MCT) | 100% | | | | | | | [symbol] | |
| 30 | Labrafac CC (MCT) | 100% | | | | | | | [symbol] | |
| 31 | Maisine 35-1 (LCM) | 100% | | | | | | | [symbol] | |
| 32 | Capmul MCM C8 (MCM) | 100% | | | | | | | [symbol] | |
| 33 | Imwitor 988 (MCM) | 100% | | | | | | | [symbol] | |
| 34 | Imwitor 380 (SCM) | 100% | | | | | | | [symbol] | |
| 35 | Lauroglycol FCC | 100% | | | | | | | [symbol] | |
| 36 | Carpryol 90 | 100% | | | | | | | [symbol] | |
| 37 | PEG 400 | 70% | EtOH | 30% | | | | | [symbol] | |
| 38 | PEG 400 | 40% | EtOH | 10% | PG | 50% | | | [symbol] | |
| 39 | Dimethylacetamide | 90% | Polysorbate 80 | 10% | | | | | [symbol] | |
| 40 | N-methyl-2-pyrrolidinone | 90% | Polysorbate 80 | 10% | | | | | [symbol] | |
| 41 | PEG 300 | 90% | Polysorbate 80 | 10% | | | | | [symbol] | |
| 42 | PEG 400 | 90% | Polysorbate 80 | 10% | | | | | [symbol] | |
| 43 | PEG 400 | 75% | Polysorbate 80 | 25% | | | | | [symbol] | |
| 44 | PG | 90% | Polysorbate 80 | 10% | | | | | [symbol] | |
| 45 | PEG 400 | 90% | Cremophor EL | 10% | | | | | [symbol] | |
| 46 | PEG 400 | 75% | Cremophor EL | 25% | | | | | [symbol] | |
| 47 | PG | 90% | Cremophor EL | 10% | | | | | [symbol] | |
| 48 | PEG 400 | 90% | TPGS | 10% | | | | | [symbol] | |
| 49 | PEG 400 | 80% | Polysorbate 80 | 10% | EtOH | 10% | | | [symbol] | |
| 50 | PG | 80% | Polysorbate 80 | 10% | EtOH | 10% | | | [symbol] | |
| 51 | PEG 400 | 60% | PG | 30% | Polysorbate 80 | 10% | | | [symbol] | |
| 52 | PEG 400 | 40% | PG | 50% | Polysorbate 80 | 10% | | | [symbol] | |
| 53 | PG | 50% | Oleic acid | 40% | Cremophor EL | 10% | | | [symbol] | |
| 54 | PEG 400 | 40% | Glycerin | 40% | Peppermint oil | 20% | | | [symbol] | |
| 55 | Dimethylacetamide | 40% | Cremophor EL | 40% | EtOH | 20% | | | [symbol] | |
| 56 | PG | 10% | Cremophor EL | 45% | Corn oil | 35% | EtOH | 10% | [symbol] | |
| 57 | PEG 400 | 10% | Cremophor EL | 45% | Corn oil | 35% | EtOH | 10% | [symbol] | |
| 58 | Neobee M-5 (MCT) | 35% | Capmul MCM C8 | 20% | Cremophor EL | 35% | EtOH | 10% | [symbol] | |
| | | | (MCM) | | | | | | | |
| 59 | Soybean oil | 35% | Maisine 35-1 (LCM) | 20% | Cremophor EL | 35% | EtOH | 10% | [symbol] | |
| 60 | Carpryol 90 | 40% | PEG 400 | 30% | Cremophor EL | 30% | | | [symbol] | |
| 61 | Labrafac CC (MCT) | 25% | Imwitor 988 (MCM) | 27% | Cremophor EL | 48% | | | [symbol] | |
| 62 | Sesame oil | 25% | Maisine 35-1 (LCM) | 27% | Cremophor EL | 48% | | | [symbol] | |
| 63 | Neobee M-5 (MCT) | 35% | Capmul MCM C8 | 20% | Polysorbate 80 | 35% | EtOH | 10% | [symbol] | |
| | | | (MCM) | | | | | | | |
| 64 | Soybean oil | 35% | Maisine 35-1 (LCM) | 20% | Polysorbate 80 | 35% | EtOH | 10% | [symbol] | |
| 65 | Carpryol 90 | 40% | PEG 400 | 30% | Polysorbate 80 | 30% | | | [symbol] | |
| 66 | Labrafac CC (MCT) | 25% | Imwitor 988 (MCM) | 27% | Polysorbate 80 | 48% | | | [symbol] | |
| 67 | Sesame oil | 25% | Maisine 35-1 (LCM) | 27% | Polysorbate 80 | 48% | | | [symbol] | |
| 68 | Dimethylacetamide | 10% | PEG 300 | 40% | | | PBS | 50% | [symbol] | [symbol] |
| 69 | Dimethylacetamide | 10% | Cremophor EL | 15% | | | PBS | 75% | [symbol] | [symbol] |
| 70 | Dimethylacetamide | 10% | Cremophor EL | 15% | EtOH | 20% | PBS | 55% | [symbol] | [symbol] |
| 71 | Cremophor EL | 10% | EtOH | 10% | | | PBS | 80% | [symbol] | [symbol] |
| 72 | Cremophor EL | 10% | EtOH | 20% | | | PBS | 70% | [symbol] | [symbol] |
| 73 | Cremophor EL | 25% | EtOH | 25% | | | PBS | 50% | [symbol] | [symbol] |
| 74 | Glycerin | 45% | Polysorbate 80 | 5% | | | PBS | 50% | [symbol] | [symbol] |
| 75 | Glycerin | 40% | Polysorbate 80 | 5% | EtOH | 5% | PBS | 50% | [symbol] | [symbol] |
| 76 | Glycerin | 30% | EtOH | 20% | | | PBS | 50% | [symbol] | [symbol] |
| 77 | PEG 300 | 30% | EtOH | 20% | | | PBS | 50% | [symbol] | [symbol] |
| 78 | PEG 400 | 20% | Glycerin | 20% | Peppermint oil | 10% | PBS | 50% | [symbol] | [symbol] |
| 79 | PG | 30% | EtOH | 20% | | | PBS | 50% | [symbol] | [symbol] |
| 80 | PEG 300 | 40% | PG | 5% | Polysorbate 80 | 5% | PBS | 50% | [symbol] | [symbol] |
| 81 | PEG 300 | 30% | PG | 15% | Polysorbate 80 | 5% | PBS | 50% | [symbol] | [symbol] |
| 82 | PEG 300 | 20% | PG | 25% | Polysorbate 80 | 5% | PBS | 50% | [symbol] | [symbol] |
| 83 | PEG 300 | 45% | Polysorbate 80 | 5% | | | PBS | 50% | [symbol] | [symbol] |
| 84 | PG | 45% | Polysorbate 80 | 5% | | | PBS | 50% | [symbol] | [symbol] |
| 85 | PEG 300 | 40% | Polysorbate 80 | 5% | EtOH | 5% | PBS | 50% | [symbol] | [symbol] |
| 86 | PG | 40% | Polysorbate 80 | 5% | EtOH | 5% | PBS | 50% | [symbol] | [symbol] |
| 87 | PEG 300 | 40% | PG | 5% | Polysorbate 80 | 5% | SGF | 50% | [symbol] | [symbol] |
| 88 | PEG 300 | 30% | PG | 15% | Polysorbate 80 | 5% | SGF | 50% | [symbol] | [symbol] |
| 89 | PEG 300 | 20% | PG | 25% | Polysorbate 80 | 5% | SGF | 50% | [symbol] | [symbol] |
| 90 | PEG 300 | 45% | Polysorbate 80 | 5% | SGF | 50% | | | [symbol] | [symbol] |
| 91 | PG | 45% | Polysorbate 80 | 5% | SGF | 50% | | | [symbol] | [symbol] |
| 92 | PEG 300 | 40% | Polysorbate 80 | 5% | EtOH | 5% | SGF | 50% | [symbol] | [symbol] |
| 93 | PG | 40% | Polysorbate 80 | 5% | EtOH | 5% | SGF | 50% | [symbol] | [symbol] |
| 94 | Labrafac CC (MCT) | 20% | PEG 300 | 30% | | | PBS | 50% | [symbol] | [symbol] |
| 95 | Soybean oil | 20% | PEG 300 | 30% | | | PBS | 50% | [symbol] | [symbol] |
| 96 | Safflower oil | 10% | PEG 300 | 40% | | | PBS | 50% | [symbol] | [symbol] |

Certain excipients in Table 1 are identified by abbreviation or trade name. The full name or generic name of these certain excipients is as follows: “PEG” refers to polyethylene glycol; “PG” refers to propylene glycol; “Polysorbate 80” refers to polyoxyethylene 20 sorbitan monooleate; “Cremphor EL” refers to polyoxyethylene 35 castor oil; “Softigen 767” refers to PEG 300 caprylic/capric glycerides; “Labrasol” refers to PEG 400 caprylic/capric glycerides; “Labrafil M 1944 CS” refers to PEG 300 linoleic glycerides; “TPGS” refers to d-tocopheryl polyethylene glycol 1000 succinate; “Povidone” refers to polyvinylpyrrolidone; “HPβCD” refers to hydroxypropyl-β-cyclodextrin; “SBEβCD” refers to sulfobutylether-β-dextrin; “RMOCD” refers to random methyl-β-cyclodextrin; “Neobee M-5 (MCT)” refers to medium-chain triglycerides; “Labrafac CC (MCT)” refers to medium-chain triglycerides; “Maisin 35-1 (LCM)” refers to glyceryl linoleate; “Capmul MCM C8 (MCM)” refers to glyceryl monocaprylate; “Imwitor 988 (MCM)” refers to glyceryl monocaprylate; “Imwitor 380 (SCM)” refers to glyceryl cocoate/citrate/acetate; “Lauroglycol FCC” refers to propylene glycol laurate; “Carpryol 90” refers to propylene glycol monocaprylate; and “SGF” refers to simulated gastric fluid.

The excipients and excipient combinations of the representative formulation screening sets set forth in Table 1 span a range of conditions (i.e., Conditions 1-96) corresponding to a variety of formulation types and solubilizing approaches. Conditions 1-6 represent pH adjustment as an approach to solubilization. Conditions 7-12 represent a co-solvent approach to solubilization. Conditions 13-18 represent a surfactant approach to solubilization. Conditions 19-22 represent a complexation approach to solubilization. Conditions 23-36 represent a lipid approach to solubilization. Conditions 57-67 represent a self-emulsifying drug delivery system (SEDDS) approach to solubilization. Conditions 94-96 represent a microemulsion approach to solubilization. Conditions 37-56 and 68-93 represent a combinational excipient approach to solubilization.

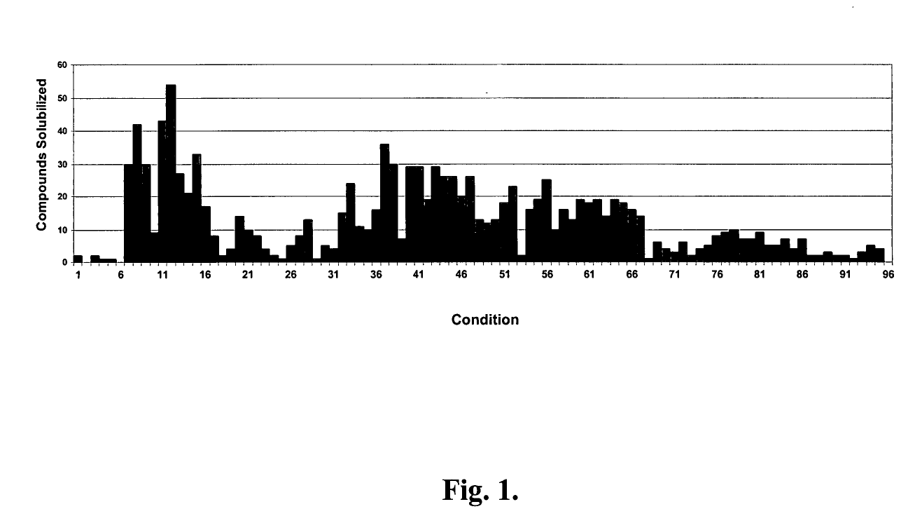

The effectiveness of a representative formulation screening set (see Table 1 excipients) in the method of the invention was determined for a set of structurally diverse small molecules (sixty-four compounds) and peptides (three) at various concentrations were tested using the high throughput formulation screening method of the invention. The compounds had a molecular weight of from about 100 to about 1000 and the peptides had a molecular weight of from about 4000 to about 5000. The formulation screening set was successful in solubilizing 100% of the compounds tested at their respective target concentrations. Although compound concentrations varied, 93% of the compounds fell within the solubility range of from 5 mg/mL to 20 mg/mL. An average of eighteen (18) distinct formulations were identified that fully solubilized the compounds during Session 3 or Session 4. FIG. 1 is a bar graph illustrating the results.

Among all excipients in the 96 formulation screening set tested for the diverse set of sixty-four (64) small molecules, polyethylene glycols, dimethylacetamide, N-methyl-2-pyrrolidinone, and Softigen 767 were found to be the best solubilizers. Polyethylene glycols (PEG300 and PEG400) are approved for human use in PO and IV applications, and have been routinely used in oral and intravenous administrations in single pharmacokinetics studies in rats and dogs. Co-solvents such as PEG400/ethanol, PEG400/ethanol/PG also work well to solubilize the poorly soluble compounds. Surfactants such as TWEEN 80 and Cremophor EL have been shown to increase the dissolution rate of a number of drugs by micellar solubilization and also improved drug's wettability. Although TWEEN 80 is known to cause hypotension among dogs during its intravenous application (see, for example, Somberg, J.C., et al., “Comparative Effects of Rapid Bolus Administration of Aqueous Amiodarone Versus 10-Minute Cordarone IV Infusion on Mean Arterial Blood Pressure in Conscious Dogs,” Cardiovasc Drugs Ther. 18:345-351, 2004; Torres-Arraut, E., et al., “Electrophysiologic Effects of Tween 80 in the Myocardium and Specialized Conduction System of the Canine Heart,” J. Electrocardiol. 17:145-151, 1984), it is still used as an excipient in the intravenous administration for dogs at low percentage (<8% by volume). Joshi, H.N., et al., “Bioavailability Enhancement of a Poorly Water-Soluble Drug by Solid Dispersion in Polyethylene Glycol-Polysorbate 80 Mixture,” Int. J Pharm. 269:251-258, 2004; Reinoso, R.F., et al., “Pharmacokinetics of E-6087, a New Anti-Inflammatory Agent, in Rats and Dogs,” Biopharm. Drug Dispos. 22:231-242, 2001; Liang, J., et al., “Preparation of Indomethacin Injection and Determination of Its Blood Concentration,” Acta Pharm. Sinica 20:22-24, 1985. The combinations of PEGs and TWEEN 80, PEGs and Cremophor EL worked well as sets of powerful solubilizing agents, which can be administrated in capsules for oral dosing or dilutes with buffer for intravenous administration. PEG300/TWEEN 80 (9/1), PEG400/Cremophor (9/1), and PEG400/Cremophor (3/1) showed the highest success rate for dissolving small molecules. The combination of PEG400 with ethanol also solubilizes thirteen (13) of eighteen (18) small molecules (see FIG. 1).

Lipid-based microemulsions and self-emulsifying drug delivery systems (SEDDS) have been shown to be an effective solubilizing approach. Self-emulsifying drug delivery systems have recently been used to improve the dissolution rate and extent of lipophilic drugs and thereby enhance their absorption. SEDDS form a fine emulsion when exposed to aqueous media under gentle agitation to provide oil-in-water emulsions that are thermodynamically stable due to the relative small volume of the dispersed oil phase, the narrow range of the oil droplet size distribution, and the polarity of the oil droplets. The digestive motility of the stomach and the intestine provide the agitation necessary for self-emulsifying when the SEDDS are orally administrated in the encapsulated soft gelatin capsules. While SEDDS typically produce emulsions with particle sizes between 100 and 300 nm, SMEDDS (self-micro-emulsifying drug delivery system) form transparent microemulsions with a particle size of less than 100 nm. The spontaneous formation of the emulsion upon the formulation release in the gastrointestinal tract afford the drug in the solubilized form, and the small droplet size enables better drug absorption via a larger interfacial surface area.

Conditions 54, 56, and 61 are representative self-emulsifying drug delivery systems. Condition 54 (PEG400/glycerine/peppermint oil=4/4/2) dissolves sixteen (16) of the sixty-four (64) small molecules; Condition 56 (PG/cremophor/corn oil/ethanol=10/45/35/10) dissolves twenty-five (25) of the sixty-four (64) small molecules; and Condition 61 (labrafac cc/imwitor 988/cremophor=25/27/48) dissolves eighteen (18) of the sixty-four (64) small molecules. (See FIG. 1).

A second representative formulation screening set is set forth in Table 2. In this set certain excipients of the set of Table 1 have been removed (e.g., DMA, NMP, RMβCD, peppermint oil, and Imwitor 380) and others have been added (e.g., peanut oil, olive oil, Solutol, Emulphor 620, Cremophor RH 40, Cremophor RH 60, vitamin E, Gelucire 44/14, and PEG600).

TABLE 2
Second Representative Formulation Screening Set.

| Con- | | | | | | | | | | | Single | IV Dog |
|---|---|---|---|---|---|---|---|---|---|---|---|---|
| dition | Excipient 1 | [%] | Excipient 2 | [%] | Excipient 3 | [%] | Excipient 4 | [%] | Oral | IV | PK | friendly |
| 1 | NaH2PO4/phosphoric | 100% | | | | | | | [symbol] | [symbol] | | [symbol] |
| | acid, pH2 | | | | | | | | | | | |
| 2 | Na3 citrate/citric acid, pH3 | 100% | | | | | | | [symbol] | [symbol] | | [symbol] |
| 3 | Na acetate/acetic acid, pH5 | 100% | | | | | | | [symbol] | [symbol] | | [symbol] |
| 4 | NaH2PO4/Na2HPO4, pH7 | 100% | | | | | | | [symbol] | [symbol] | | [symbol] |
| 5 | NaH2PO4/Na2HPO4, pH8 | 100% | | | | | | | [symbol] | [symbol] | | [symbol] |
| 6 | Na2CO3/HCl, pH10 | 100% | | | | | | | [symbol] | [symbol] | | [symbol] |
| 7 | Solutol | 10% | | | | | PBS | 90% | [symbol] | [symbol] | | [symbol] |
| 8 | Emulphor 620 | 10% | EtOH | 10% | | | PBS | 80% | [symbol] | [symbol] | [symbol] | [symbol] |
| 9 | Glycerin | 40% | EtOH | 10% | | | PBS | 50% | [symbol] | [symbol] | [symbol] | [symbol] |
| 10 | PG | 40% | EtOH | 10% | | | PBS | 50% | [symbol] | [symbol] | [symbol] | [symbol] |
| 11 | PG | 40% | Solutol | 5% | EtOH | 5% | PBS | 50% | [symbol] | [symbol] | [symbol] | [symbol] |
| 12 | PG | 45% | Solutol | 5% | | | PBS | 50% | [symbol] | [symbol] | | [symbol] |
| 13 | PEG 300 | 20% | Glycerin | 20% | Solutol | 10% | PBS | 50% | [symbol] | [symbol] | | [symbol] |
| 14 | PEG 300 | 20% | PG | 25% | Solutol | 5% | PBS | 50% | [symbol] | [symbol] | | [symbol] |
| 15 | PEG 300 | 30% | EtOH | 20% | | | PBS | 50% | [symbol] | [symbol] | [symbol] | [symbol] |
| 16 | PEG 300 | 30% | PG | 15% | Solutol | 5% | PBS | 50% | [symbol] | [symbol] | | [symbol] |
| 17 | PEG 300 | 40% | PG | 5% | Solutol | 5% | PBS | 50% | [symbol] | [symbol] | | [symbol] |
| 18 | PEG 300 | 40% | Solutol | 5% | EtOH | 5% | PBS | 50% | [symbol] | [symbol] | [symbol] | [symbol] |
| 19 | PEG 300 | 45% | Solutol | 5% | | | PBS | 50% | [symbol] | [symbol] | | [symbol] |
| 20 | HPβCD | 40% | | | | | | | [symbol] | [symbol] | | [symbol] |
| 21 | SBEβCD | 40% | | | | | | | [symbol] | [symbol] | | [symbol] |
| 22 | Glycerin | 40% | Polysorbate 80 | 5% | EtOH | 5% | PBS | 50% | [symbol] | [symbol] | [symbol] | |
| 23 | Glycerin | 45% | Polysorbate 80 | 5% | | | PBS | 50% | [symbol] | [symbol] | | |
| 24 | PG | 40% | Polysorbate 80 | 5% | EtOH | 5% | PBS | 50% | [symbol] | [symbol] | [symbol] | |
| 25 | PG | 45% | Polysorbate 80 | 5% | | | PBS | 50% | [symbol] | [symbol] | | |
| 26 | PEG 300 | 20% | PG | 25% | Polysorbate 80 | 5% | PBS | 50% | [symbol] | [symbol] | | |
| 27 | PEG 300 | 30% | PG | 15% | Polysorbate 80 | 5% | PBS | 50% | [symbol] | [symbol] | | |
| 28 | PEG 300 | 40% | PG | 5% | Polysorbate 80 | 5% | PBS | 50% | [symbol] | [symbol] | | |
| 29 | PEG 300 | 40% | Polysorbate 80 | 5% | EtOH | 5% | PBS | 50% | [symbol] | [symbol] | [symbol] | |
| 30 | PEG 300 | 45% | Polysorbate 80 | 5% | | | PBS | 50% | [symbol] | [symbol] | | |
| 31 | Cremophor RH 40 | 10% | | | | | PBS | 90% | [symbol] | [symbol] | | |
| 32 | Cremophor RH 60 | 10% | | | | | PBS | 90% | [symbol] | [symbol] | | |
| 33 | Polysorbate 80 | 10% | | | | | PBS | 90% | [symbol] | [symbol] | | |
| 34 | Vitamin E | 6% | TPGS | 3% | Solutol | 1% | PBS | 90% | [symbol] | [symbol] | | |
| 35 | Cremophor EL | 10% | EtOH | 10% | | | PBS | 80% | [symbol] | [symbol] | [symbol] | |
| 36 | Cremophor EL | 10% | EtOH | 20% | | | PBS | 70% | [symbol] | [symbol] | [symbol] | |
| 37 | Cremophor EL | 25% | EtOH | 25% | | | PBS | 50% | [symbol] | [symbol] | [symbol] | |
| 38 | Labrafac CC (MCT) | 20% | PEG 300 | 30% | | | PBS | 50% | [symbol] | [symbol] | | |
| 39 | Soybean oil | 20% | PEG 300 | 30% | | | PBS | 50% | [symbol] | [symbol] | | |
| 40 | Safflower oil | 10% | PEG 300 | 40% | | | PBS | 50% | [symbol] | [symbol] | | |
| 41 | PEG 400 | 70% | EtOH | 30% | | | | | [symbol] | | [symbol] | |
| 42 | PEG 400 | 40% | EtOH | 10% | PG | 50% | | | [symbol] | | [symbol] | |
| 43 | PEG 400 | 75% | Polysorbate 80 | 25% | | | | | [symbol] | | | |
| 44 | PEG 400 | 75% | TPGS | 25% | | | | | [symbol] | | | |
| 45 | PEG 400 | 75% | Solutol | 25% | | | | | [symbol] | | | |
| 46 | PEG 400 | 75% | Cremophor EL | 25% | | | | | [symbol] | | | |
| 47 | PEG 400 | 75% | Cremophor RH 40 | 25% | | | | | [symbol] | | | |
| 48 | PEG 400 | 75% | Cremophor RH 60 | 25% | | | | | [symbol] | | | |
| 49 | PEG 400 | 75% | Gelucire 44/14 | 25% | | | | | [symbol] | | | |
| 50 | PEG 400 | 75% | Labrasol | 25% | | | | | [symbol] | | | |
| 51 | PG | 75% | TPGS | 25% | | | | | [symbol] | | | |
| 52 | PEG 400 | 80% | Polysorbate 80 | 10% | EtOH | 10% | | | [symbol] | | [symbol] | |
| 53 | PEG 400 | 80% | TPGS | 10% | EtOH | 10% | | | [symbol] | | [symbol] | |
| 54 | PEG 400 | 60% | PG | 30% | TPGS | 10% | | | [symbol] | | | |
| 55 | PEG 400 | 40% | PG | 50% | TPGS | 10% | | | [symbol] | | | |
| 56 | PEG 400 | 40% | Glycerin | 40% | TPGS | 20% | | | [symbol] | | | |
| 57 | PG | 50% | Oleic acid | 40% | Cremophor EL | 10% | | | [symbol] | | | |
| 58 | PG | 10% | Cremophor EL | 45% | Corn oil | 35% | EtOH | 10% | [symbol] | | [symbol] | |
| 59 | PEG 400 | 10% | Cremophor EL | 45% | Corn oil | 35% | EtOH | 10% | [symbol] | | [symbol] | |
| 60 | Neobee M-5 (MCT) | 35% | Capmul MCM C8 | 20% | Cremophor EL | 35% | EtOH | 10% | [symbol] | | [symbol] | |
| | | | (MCM) | | | | | | | | | |
| 61 | Soybean oil | 35% | Maisine 35-1 (LCM) | 20% | Cremophor EL | 35% | EtOH | 10% | [symbol] | | [symbol] | |
| 62 | Carpryol 90 | 40% | PEG 400 | 30% | Cremophor EL | 30% | | | [symbol] | | | |
| 63 | Labrafac CC (MCT) | 25% | Imwitor 988 (MCM) | 27% | Cremophor EL | 48% | | | [symbol] | | | |
| 64 | Sesame oil | 25% | Maisine 35-1 (LCM) | 27% | Cremophor EL | 48% | | | [symbol] | | | |
| 65 | Neobee M-5 (MCT) | 35% | Capmul MCM C8 | 20% | Polysorbate 80 | 35% | EtOH | 10% | [symbol] | | [symbol] | |
| | | | (MCM) | | | | | | | | | |
| 66 | Soybean oil | 35% | Maisine 35-1 (LCM) | 20% | Polysorbate 80 | 35% | EtOH | 10% | [symbol] | | [symbol] | |
| 67 | Carpryol 90 | 40% | PEG 400 | 30% | Polysorbate 80 | 30% | | | [symbol] | | | |
| 68 | Labrafac CC (MCT) | 25% | Imwitor 988 (MCM) | 27% | Polysorbate 80 | 48% | | | [symbol] | | | |
| 69 | Sesame oil | 25% | Maisine 35-1 (LCM) | 27% | Polysorbate 80 | 48% | | | [symbol] | | | |
| 70 | TPGS | 10% | | | | | | | [symbol] | | | |
| 71 | Povidone | 10% | | | | | | | [symbol] | | | |
| 72 | Peanut oil | 100% | | | | | | | [symbol] | | | |
| 73 | Corn oil | 100% | | | | | | | [symbol] | | | |
| 74 | Safflower oil | 100% | | | | | | | [symbol] | | | |
| 75 | Sesame oil | 100% | | | | | | | [symbol] | | | |
| 76 | Soybean oil | 100% | | | | | | | [symbol] | | | |
| 77 | Olive oil | 100% | | | | | | | [symbol] | | | |
| 78 | Vitamin E | 100% | | | | | | | [symbol] | | | |
| 79 | Oleic acid | 100% | | | | | | | [symbol] | | | |
| 80 | Neobee M-5 (MCT) | 100% | | | | | | | [symbol] | | | |
| 81 | Labrafac CC (MCT) | 100% | | | | | | | [symbol] | | | |
| 82 | Maisine 35-1 (LCM) | 100% | | | | | | | [symbol] | | | |
| 83 | Capmul MCM C8 (MCM) | 100% | | | | | | | [symbol] | | | |
| 84 | Imwitor 988 (MCM) | 100% | | | | | | | [symbol] | | | |
| 85 | Lauroglycol FCC | 100% | | | | | | | [symbol] | | | |
| 86 | Carpryol 90 | 100% | | | | | | | [symbol] | | | |
| 87 | PEG 300 | 100% | | | | | | | [symbol] | | | |
| 88 | PEG 400 | 100% | | | | | | | [symbol] | | | |
| 89 | PEG 600 | 100% | | | | | | | [symbol] | | | |
| 90 | PG | 100% | | | | | | | [symbol] | | | |
| 91 | Glycerin | 100% | | | | | | | [symbol] | | | |
| 92 | Softigen 767 | 100% | | | | | | | [symbol] | | | |
| 93 | Labrasol | 100% | | | | | | | [symbol] | | | |
| 94 | Labrafil M 1944 CS | 100% | | | | | | | [symbol] | | | |
| 95 | Polysorbate 80 | 100% | | | | | | | [symbol] | | [symbol] | |
| 96 | Cremophor EL | 100% | | | | | | | [symbol] | | [symbol] | |

Certain excipients in Table 2 are identified by abbreviation or trade name not previously identified in Table 1. The full name or generic name of these certain excipients is as follows: “Solutol” refers to a composition that is 70% lipophilic consisting of polyglycol mono- and diesters of 12-hydroxystearic acid and 30% hydrophilic consisting of polyethylene glycol (BASF), “Emulphor 620” refers to castor oil ethoxylate (30) CAS No. 61791-12-6 (Rhodia), “Cremophor RH 40” refers to polyoxy 40 hydrogenated castor oil (BASF), “Cremophor RH 60” refers to polyoxy 60 hydrogenated castor oil (BASF), and “Gelucire 44/14” refers to a mixture of mono-, di-, and triglycerides and mono- and di-fatty acid esters of PEG1500. Gelucire44/14 is synthesized by an alcoholysis/esterification using palm kernel oil and PEG1500, and the main fatty acid is lauric acid

The present invention provides a solution to the problem of the preformulation bottleneck in drug discovery and development. Using liquid handling and imaging hardware, informatics, and special plasticware, the present invention provides scaleable high-throughput methods for simultaneously assessing compound solubility and solution stability.

In one embodiment, the screening method of the invention uses a primary formulation screen set that is a broad-range, multi-component ninety-six (96)-condition screen composed of biocompatible excipients. The method and screen set employs basic solubilization approaches for both oral and parenteral administrations: pH adjustment, cosolvent, oil, micelle, organic solvent/surfactant, complexation, and self-emulsifying drug delivery systems. In the method, the screen set is used as the initial screen of all compounds to assess their solubility, crystallizability, and compatibility with various formulations in order to design compound specific optimization screening sets.

The high throughput formulation screening method of the invention is a rapid and cost efficient way to identify biocompatible dosing solutions for small molecules. The method utilizes minute amounts of test compound and provides solution/crystallization stability time courses. Information gained from the method can be applied to rapidly identify dosing solutions for initial animal studies as well as recrystallization conditions for polymorph studies.

The method of the invention is useful for screening small molecules, peptides, proteins, fatty acids, and proteins for their solubility and solution stability in various formulations. The method of the invention is useful for screening small molecules, peptides, proteins, fatty acids, and proteins for their applicability as solid dispersion and nanoparticle formulations. The method of the invention is also useful in identifying polymorphs and solvates formed as a result of interaction with an excipient. The method of the invention is useful in generating compound specific screens. The method of the invention is useful for aiding in final drug product formulation design.

The following example is provided for the purpose of illustrating, not limiting, the invention.

### EXAMPLES

### Example 1

### Description of Materials and Methods for a Representative Formulation Screen

In this example, the materials and methods used for a representative formulation screening method of the invention is described. Formulation screening results for a representative compound, 3,4,4′-trichlorocarbanilide (TCC, also referred to herein as “triclocarban”), are provided.

Representative test compound: 3,4,4′-trichlorocarbanilide (TCC) was obtained from Sigma-Aldrich (St. Louis, Mo.).

Excipients: All chemicals were obtained from either Sigma-Aldrich (St. Louis, Mo.) or Spectrum Chemicals (Gardena, Calif.) except for the following: HPβCD (degree of substitution 0.8) and RMβCD (degree of substitution 1.8) were purchased from Wacker Chemical Corporation (Adrian, Mich.); TPGS was obtained from Eastman Chemical (Kingsport, Tenn.); Captex 355 EP and Capmul MCM C8 were obtained from Abitec Corporation (Janesville, Wis.); Labrasol, Labrafil M 1944 CS, Labrafac CC, Maisine 35-1, Lauroglycol FCC, Carpryol 90 were obtained from Gattefosse (Paramus, N.J.); Softigen 767, Imwitor 988, Imwitor 380 were obtained from Sasol (Springfield, N.J.). The compositions of the 96 screening formulations are summarized in Table 1.

Screening materials: the 96-well storage block and sealing mat was obtained from Matrix Technologies (Hudson, N.H.). The 96-well plate was obtained from Emerald BioSystems (Bainbridge Island, Wash.). The sealing tape was purchase from Henkel Consumer Adhesives (Avon, Ohio).

Instrumentation: MATRIX MAKER liquid handler (Emerald BioSystems, Bainbridge Island, Wash.) was used to prepare excipient stock solution. This instrument includes thirty (30) independent positive displacement pumps, sixty (60) on board stock solutions, specifically designed for accurately dispensing highly viscous excipients (e.g., Cremophor EL) as well as high vapor pressure solvents (e.g., methanol). DROP MAKER micro-scale liquid handler (Emerald BioSystems, Bainbridge Island, Wash.) was used to dispense compound solutions to the assay plate. Crystal monitor workstation (Emerald BioSystems, Bainbridge Island, Wash.) combines a Leica stereomicroscope, a motorized stage and a digital camera to capture and store high resolution images of individual wells at a rate of twelve 96-well plates per hour. CRYSTAL MINER is the Oracle- or SQL Server-compatible relational database software that drives the individual hardware components and manages the data generated.

Preparation of Representative Screen Formulation Stock Solutions: All stock solutions were prepared with ultrapure ASTM Type I water and sterile-filtered using a 0.22 micron filter. The solutions were then attached to the MATRIX MAKER liquid-handling robot for sterile path, positive pressure dispensation into an SBS (Society for Biomolecular Screening) standard-sized 96 deep well plates. The screening formulations are defined in the CRYSTAL MINER software, which drives the MATRIX MAKER and allows for the rapid development of refinement screens for the optimization of lead hits. Once the formulations have been dispensed, the plate is sealed and centrifuged at 2000 rpm to ensure that each well contains a homogeneous solution.

### Formulation Screening Method

The test compound is completely dissolved in a high vapor pressure organic solvent such as methanol at 10 mg/mL. The DROP MAKER micro-scale liquid handler then dispenses 5 μL of the compound solution into a 96-well SBS format plate. The plated solutions were evaporated to dryness leaving 50 μg of dry compound in each well. Images were then captured of each well by the CRYSTAL MONITOR workstation (Session 1). Then 5 μL of each formulation screening solution were added to separate wells and images were captured for each well (Session 2) as above. Following image capture, the plate was heated to 45° C. for 30 minutes, then agitated for 10 minutes at room temperature during cool down. Additional images are captured on day one (t=1, Session 3) and day three (t=3, Session 4). Images and data annotation are exported to Excel for final report generation.

Scaled-Up Procedure: Selected conditions from the initial screening were scaled up 200-fold by volume (from 5 μL to 1 mL). The test compound, 10 mg TCC, was mixed with 1 mL of formulation stock solutions in a 4-mL glass vial, sonicated for 5 minutes, and then shaken overnight at room temperature. The extent of the compound's dissolution was then determined and classified as not dissolved, partially dissolved, precipitated, or completely dissolved, and recorded on the following day for each formulation.

### Results

Triclocarban (TCC) is an antibacterial agent commonly found in household products such as toothpastes and antibacterial soaps. Triclocarban is unionized at pH below 11, has a melting point of 255 to 256° C., its log P_{octanol/water} is 4.9. Moffat, A.C., et al., Clarke's Analysis of Drugs and Poisons, Pharmaceutical Press, London, 2004. TCC is lipophilic and water-insoluble, but is soluble in various organic solvents such as acetone and, to some extent, propylene glycol. The intrinsic solubility (S₀) of triclocarban in pure water at room temperature has been determined to be less than 50 ng/ml. Loftsson, T., et al., “Cyclodextrin Solubilization of the Antibacterial Agents Triclosan and Triclocarban: Effect of Ionization and Polymers,” J Incl. Phenom. Macroc. Chem. 52:109-117, 2005; Duan, M. S., et al., “Cyclodextrin Solubilization of the Antibacterial Agents Triclosan and Triclocarban: Formation of Aggregates and Higher-Order Complexes,” Int. J Pharm. 297:213-222, 2005. The solubility of triclocarban was improved to about 5 mg/mL with 20% RMbCD with additives.

The solubility of triclocarban in the formulations screening set (96 conditions) was obtained after the high throughput screening at target solubility of 10 mg/mL by the method described above:

each of PEG 300, PEG 400, Polysorbate 80, dimethylacetamide, Capmul MCM C8, and Imwitor 988 were able to dissolve TCC completely at 10 mg/mL;

each of N-methyl-2-pyrrolidinone, Cremophor EL, Softigen 767, Labrasol, Labrafil M 1944 CS, peppermint oil, lauroglycol FCC, and Carpryol 90 dissolved TCC at almost 10 mg/mL;

adjustment of pH to between 2 and 10 had no effect on solubilizing TCC;

neither glycerine, safflower oil, sesame oil, nor 20% povidone had any effect on solubilizing TCC;

each of 20% TPGS, HPbCD, RMbCD, corn oil, soybean oil, oleic acid, neobee M-5, labrafac CC, maisine 35-1, imwitor 380 was partially dissolved TCC at the target 10 mg/mL;

each of cosolvents PEG 400/EtOH (7/3), PEG 400/EtOH/PG (4/1/5), NMP/polysorbate 80 (9/1), PEG 300/polysorbate 80 (9/1), PEG 400/polysorbate (3/1), PEG 400/polysorbate 80/EtOH (8/1/1) easily dissolved TCC at 10 mg/mL;

each of the self-emulsifying drug delivery systems carpryol 90/PEG 400/cremophor EL (4/3/3), labrafac CC/imwitor 988/cremophor (25/27/48), soybean oil/maisine 35-1/polysorbate 80/EtOH (35/20/35/10), and sesame oil/maisine 35-1/polysorbate 80 (25/27/48) dissolved TCC at 10 mg/mL; and

each of the intravenous (IV) dosing solutions PEG 300/polysorbate 80/SGF (45/5/50) and PEG 300/polysorbate 80/EtOH/SGF (40/5/5/50) nearly completely dissolved TCC at 10 mg/mL.

The formulation screening results for TCC using a representative formulation screening set described in Table 1 are summarized in Table 3.

TABLE 3
High throughput formulation screening results for TCC.

| Condition | Session 1 | Session 2 | Session 3 | Session 4 |
|---|---|---|---|---|
| 1 | Compound Present | Not Dissolved | Not Dissolved | Not Dissolved |
| 2 | Compound Present | Not Dissolved | Not Dissolved | Not Dissolved |
| 3 | Compound Present | Not Dissolved | Not Dissolved | Not Dissolved |
| 4 | Compound Present | Not Dissolved | Not Dissolved | Not Dissolved |
| 5 | Compound Present | Not Dissolved | Not Dissolved | Not Dissolved |
| 6 | Compound Present | Not Dissolved | Not Dissolved | Not Dissolved |
| 7 | Compound Present | Completely Dissolved | Completely Dissolved | Completely Dissolved |
| 8 | Compound Present | Completely Dissolved | Completely Dissolved | Completely Dissolved |
| 9 | Compound Present | Mostly Dissolved | Mostly Dissolved | Precipitate |
| 10 | Compound Present | Not Dissolved | Not Dissolved | Not Dissolved |
| 11 | Compound Present | Completely Dissolved | Completely Dissolved | Precipitate |
| 12 | Compound Present | Mostly Dissolved | Mostly Dissolved | Mostly Dissolved |
| 13 | Compound Present | Completely Dissolved | Completely Dissolved | Completely Dissolved |
| 14 | Compound Present | Mostly Dissolved | Mostly Dissolved | Completely Dissolved |
| 15 | Compound Present | Mostly Dissolved | Mostly Dissolved | Mostly Dissolved |
| 16 | Compound Present | Mostly Dissolved | Mostly Dissolved | Mostly Dissolved |
| 17 | Compound Present | Mostly Dissolved | Completely Dissolved | Precipitate |
| 18 | Compound Present | Not Dissolved | Partially Dissolved | Partially Dissolved |
| 19 | Compound Present | Not Dissolved | Not Dissolved | Partially Dissolved |
| 20 | Compound Present | Not Dissolved | Partially Dissolved | Mostly Dissolved |
| 21 | Compound Present | Not Dissolved | Not Dissolved | Not Dissolved |
| 22 | Compound Present | Not Dissolved | Partially Dissolved | Partially Dissolved |
| 23 | Compound Present | Partially Dissolved | Partially Dissolved | Partially Dissolved |
| 24 | Compound Present | Not Dissolved | Not Dissolved | Not Dissolved |
| 25 | Compound Present | Not Dissolved | Not Dissolved | Partially Dissolved |
| 26 | Compound Present | Partially Dissolved | Partially Dissolved | Partially Dissolved |
| 27 | Compound Present | Mostly Dissolved | Mostly Dissolved | Mostly Dissolved |
| 28 | Compound Present | Partially Dissolved | Partially Dissolved | Partially Dissolved |
| 29 | Compound Present | Partially Dissolved | Precipitate | Precipitate |
| 30 | Compound Present | Partially Dissolved | Mostly Dissolved | Precipitate |
| 31 | Compound Present | Not Dissolved | Partially Dissolved | Partially Dissolved |
| 32 | Compound Present | Completely Dissolved | Completely Dissolved | Completely Dissolved |
| 33 | Compound Present | Mostly Dissolved | Completely Dissolved | Completely Dissolved |
| 34 | Compound Present | Not Dissolved | Partially Dissolved | Mostly Dissolved |
| 35 | Compound Present | Mostly Dissolved | Mostly Dissolved | Precipitate |
| 36 | Compound Present | Mostly Dissolved | Mostly Dissolved | Mostly Dissolved |
| 37 | Compound Present | Mostly Dissolved | Completely Dissolved | Completely Dissolved |
| 38 | Compound Present | Completely Dissolved | Completely Dissolved | Completely Dissolved |
| 39 | Compound Present | Completely Dissolved | Precipitate | Precipitate |
| 40 | Compound Present | Completely Dissolved | Completely Dissolved | Completely Dissolved |
| 41 | Compound Present | Completely Dissolved | Completely Dissolved | Completely Dissolved |
| 42 | Compound Present | Mostly Dissolved | Mostly Dissolved | Mostly Dissolved |
| 43 | Compound Present | Mostly Dissolved | Completely Dissolved | Completely Dissolved |
| 44 | Compound Present | Mostly Dissolved | Mostly Dissolved | Precipitate |
| 45 | Compound Present | Completely Dissolved | Completely Dissolved | Completely Dissolved |
| 46 | Compound Present | Mostly Dissolved | Completely Dissolved | Completely Dissolved |
| 47 | Compound Present | Mostly Dissolved | Mostly Dissolved | Precipitate |
| 48 | Compound Present | Not Dissolved | Mostly Dissolved | Completely Dissolved |
| 49 | Compound Present | Completely Dissolved | Completely Dissolved | Completely Dissolved |
| 50 | Compound Present | Partially Dissolved | Mostly Dissolved | Mostly Dissolved |
| 51 | Compound Present | Mostly Dissolved | Mostly Dissolved | Mostly Dissolved |
| 52 | Compound Present | Partially Dissolved | Mostly Dissolved | Mostly Dissolved |
| 53 | Compound Present | Not Dissolved | Not Dissolved | Not Dissolved |
| 54 | Compound Present | Partially Dissolved | Mostly Dissolved | Precipitate |
| 55 | Compound Present | Mostly Dissolved | Mostly Dissolved | Precipitate |
| 56 | Compound Present | Mostly Dissolved | Mostly Dissolved | Mostly Dissolved |
| 57 | Compound Present | Mostly Dissolved | Mostly Dissolved | Mostly Dissolved |
| 58 | Compound Present | Mostly Dissolved | Mostly Dissolved | Mostly Dissolved |
| 59 | Compound Present | Mostly Dissolved | Mostly Dissolved | Completely Dissolved |
| 60 | Compound Present | Completely Dissolved | Completely Dissolved | Completely Dissolved |
| 61 | Compound Present | Completely Dissolved | Completely Dissolved | Completely Dissolved |
| 62 | Compound Present | Not Dissolved | Mostly Dissolved | Completely Dissolved |
| 63 | Compound Present | Mostly Dissolved | Mostly Dissolved | Mostly Dissolved |
| 64 | Compound Present | Mostly Dissolved | Completely Dissolved | Completely Dissolved |
| 65 | Compound Present | Mostly Dissolved | Mostly Dissolved | Mostly Dissolved |
| 66 | Compound Present | Mostly Dissolved | Mostly Dissolved | Mostly Dissolved |
| 67 | Compound Present | Mostly Dissolved | Completely Dissolved | Completely Dissolved |
| 68 | Compound Present | Not Dissolved | Not Dissolved | Not Dissolved |
| 69 | Compound Present | Not Dissolved | Not Dissolved | Not Dissolved |
| 70 | Compound Present | Not Dissolved | Partially Dissolved | Partially Dissolved |
| 71 | Compound Present | Not Dissolved | Not Dissolved | Not Dissolved |
| 72 | Compound Present | Not Dissolved | Not Dissolved | Not Dissolved |
| 73 | Compound Present | Not Dissolved | Not Dissolved | Not Dissolved |
| 74 | Compound Present | Not Dissolved | Not Dissolved | Not Dissolved |
| 75 | Compound Present | Not Dissolved | Not Dissolved | Not Dissolved |
| 76 | Compound Present | Not Dissolved | Not Dissolved | Not Dissolved |
| 77 | Compound Present | Not Dissolved | Not Dissolved | Partially Dissolved |
| 78 | Compound Present | Not Dissolved | Not Dissolved | Not Dissolved |
| 79 | Compound Present | Not Dissolved | Not Dissolved | Not Dissolved |
| 80 | Compound Present | Not Dissolved | Not Dissolved | Not Dissolved |
| 81 | Compound Present | Not Dissolved | Not Dissolved | Not Dissolved |
| 82 | Compound Present | Not Dissolved | Not Dissolved | Not Dissolved |
| 83 | Compound Present | Not Dissolved | Not Dissolved | Partially Dissolved |
| 84 | Compound Present | Not Dissolved | Not Dissolved | Not Dissolved |
| 85 | Compound Present | Not Dissolved | Not Dissolved | Partially Dissolved |
| 86 | Compound Present | Not Dissolved | Not Dissolved | Not Dissolved |
| 87 | Compound Present | Not Dissolved | Not Dissolved | Not Dissolved |
| 88 | Compound Present | Not Dissolved | Not Dissolved | Not Dissolved |
| 89 | Compound Present | Not Dissolved | Not Dissolved | Not Dissolved |
| 90 | Compound Present | Not Dissolved | Not Dissolved | Mostly Dissolved |
| 91 | Compound Present | Not Dissolved | Not Dissolved | Not Dissolved |
| 92 | Compound Present | Not Dissolved | Not Dissolved | Mostly Dissolved |
| 93 | Compound Present | Not Dissolved | Not Dissolved | Not Dissolved |
| 94 | Compound Present | Not Dissolved | Not Dissolved | Not Dissolved |
| 95 | Compound Present | Not Dissolved | Not Dissolved | Partially Dissolved |
| 96 | Compound Present | Not Dissolved | Not Dissolved | Not Dissolved |

Scale-up to 1 mL formulation stock solutions provided results identical to the high throughput screening method conducted at 5 μL. Scale-up demonstrated a strong correlation between the typical laboratory scale (about 1 mL) and micro-scale (5 μL) was obtained for the high throughput formulation screening method.

While illustrative embodiments have been illustrated and described, it will be appreciated that various changes can be made therein without departing from the spirit and scope of the invention.

1. A method for screening compounds for solubility, comprising:
(a) dispensing a quantity of a compound into each well of a plurality of wells to provide a plurality of wells each containing a quantity of the compound;
(b) adding a different excipient to each well; and
(c) observing the solubility of the compound in the excipient in each well.

2. The method of claim 1, wherein the compound is a therapeutic drug candidate selected from the group consisting of a small molecule, protein, peptide, oligonucleotide, and nucleotide.

3. The method of claim 1, wherein dispensing a compound into a plurality of wells comprises dispensing from about 5 to about 500 μg of the compound to the well.

4. The method of claim 1, wherein dispensing a compound into a plurality of wells comprises dispensing about 50 μg of the compound to the well.

5. The method of claim 1, wherein adding an excipient to each well comprises adding from about 1 to about 200 μL of excipient to each well.

6. The method of claim 1, wherein adding an excipient to each well comprises adding about 5 μL of excipient to each well.

7. The method of claim 1, wherein observing the solubility of the compound in each well comprises capturing an image of each well.

8. The method of claim 1 further comprising heating the plurality of wells to a predetermined temperature and for a predetermined time after observing the solubility of the compound in each well.

9. The method of claim 8, wherein the solubility of the compound is observed after heating the plurality of wells to a predetermined temperature and for a predetermined time.

10. The method of claim 9, wherein the solubility of the compound is observed at one or more predetermined times after heating the plurality of wells.

11. The method of claim 1, wherein the excipient is representative of a solubilization approach selected from the group consisting of pH adjustment, co-solvent, oil, micelle, organic solvent/surfactant, complexation, microemulsion, self-emulsifying drug delivery system, and combinations thereof.

12. The method of claim 1, wherein the excipient is selected from the excipients set forth in Table 1.

13. The method of claim 1, wherein the excipient is selected from the excipients set forth in Table 2.

14. A method for screening compounds for solubility, comprising:
(a) dispensing a quantity of a compound into each well of a plurality of wells to provide a plurality of wells each containing a quantity of the compound;
(b) capturing an image of each well containing the compound;
(c) adding a different excipient to each well;
(d) observing the solubility of the compound in the excipient in each well by capturing a second image of each well;
(e) heating the plurality of wells to a predetermined temperature for a predetermined time;
(f) observing the solubility of the compound in the excipient in each well by capturing a third image of each well;
(g) observing the solubility of the compound in the excipient in each well by capturing a fourth image of each well after a predetermined period of time; and
(h) comparing the first, second, third, and fourth images to evaluate the solubility of the compound in each well.

15. The method of claim 14, further comprising observing the solubility of the compound in the excipient in each well by capturing a fifth image of each well after a predetermined period of time and comparing the first, second, third, fourth, and fifth images to evaluate the solubility of the compound in each well.

16. The method of claim 14, wherein the compound is a therapeutic drug candidate selected from the group consisting of a small molecule, protein, peptide, oligonucleotide, and nucleotide.

17. The method of claim 14, wherein the excipient is representative of a solubilization approach selected from the group consisting of pH adjustment, co-solvent, oil, micelle, organic solvent/surfactant, complexation, microemulsion, and self-emulsifying drug delivery system.

18. A set of formulation solutions comprising excipients representative of a solubilization approach selected from the group consisting of pH adjustment, co-solvent, oil, micelle, organic solvent/surfactant, complexation, microemulsion, and self-emulsifying drug delivery system.

19. The set of claim 18, wherein the excipients are selected from the group consisting of excipients set forth in Table 1.

20. The set of claim 18, wherein the excipients are selected from the group consisting of excipients set forth in Table 2.
